(19) United States
(12) Patent Application Publication
(10) Pub. No.: US 2018/0325831 A1
(43) Pub. Date: Nov. 15, 2018

(54) NANO COMPLEX MATERIAL, DRUG CARRIER COMPRISING SAME, CONTRAST AGENT COMPRISING SAME, AND METHOD FOR PREPARING NANO COMPLEX MATERIAL

(71) Applicant: RESEARCH COOPERATION FOUNDATION OF YEUNGNAM UNIVERSITY, Gyeongsangbuk-do (KR)

(72) Inventor: Jeong Hoon BYEON, Gyeongsangbuk-do (KR)

(21) Appl. No.: 15/979,991

(22) Filed: May 15, 2018

(30) Foreign Application Priority Data
May 15, 2017 (KR) 10-2017-0059810

(51) Int. Cl. A61K 9/51; A61K 49/18

(52) U.S. Cl. CPC A61K 9/5115; A61K 49/1824

(57) ABSTRACT

The present disclosure relates to a nano complex material, a drug carrier including the same, a contract agent including the same, and a method for preparing the nano complex material, and the nano complex material of the present disclosure consists of a support and ionic particles and thus can exhibit excellent light absorbance, magnetic absorbance, storage stability, functionality as a contrast agent, and drug delivery performance, as well as low cytotoxicity.

### CROSS-REFERENCE TO RELATED APPLICATION

This application claims priority to and the benefit of Korean Patent Application No. 10-2017-0059810, filed on May 15, 2017, the disclosure of which is incorporated herein by reference in its entirety.

### BACKGROUND

### 1. Field of the Invention

The present disclosure relates to a nano complex material, a drug carrier including the same, a contract agent including the same, and a method for preparing the nano complex material.

### 2. Discussion of Related Art

Due to the rapid development of nanotechnology, nanoparticles having various functionalities have been developed. These nanoparticles are applied to various uses such as contrast agents in the biotechnology field, drug carriers for delivering genes, photothermal therapeutic agents, or the like.

Conventional nanoparticles are mostly formed with a single component, but since such nanoparticles perform only a single function, their application fields and uses are limited. As a result, recently, studies on hetero-nanoparticles in which various functionalities can be realized by combining different types of components are actively being conducted. Since the hetero-nanoparticles exhibit various structures and interface properties by including a combination of different components, they can be used in many ways in various fields. Particularly, the hetero-nanoparticles are used for simultaneously performing treatment and diagnosis in the medical field.

Meanwhile, among the components constituting the hetero-nanoparticles, gold and iron are well known as non-proinflammatory agents. Because of such characteristics, conventionally, nanoparticles whose main components are gold and iron are used as contrast agents or thermotherapeutic agents. For example, nanoparticles whose main component is iron are used in areas of the biotechnology field such as magnetic resonance imaging (MRI) devices, drug targeting, cell transformation, or the like. In addition, nanoparticles whose main component is gold are used in computed tomography (CT) devices or the like. Recently, although hetero-nanoparticles in which gold and iron are combined have been used as a contrast agent in MRI-CT dual-mode imaging devices, improvements are required in terms of performance and reliability.

Conventionally, various wet chemistry formulations based on the suspension of solid particles were used as a method for preparing the hetero-nanoparticles. However, various wet chemistry formulations are complex and involve many steps. Therefore, in conventional wet chemistry formulation, it is difficult to quantitatively combine different components or transform particles to have physical properties suitable for their intended purposes.

In particular, seed-mediated chemistry formulation is well known among the conventional wet chemistry formulations. Seed-mediated chemistry formulation is a process of preparing hetero-nanoparticles by using one metal as a seed and growing another metal on the seed. The seed-mediated chemistry formulation uses a reducing agent in order to grow another metal component on the seed, but the reducing agent is toxic in most cases. Therefore, the seed-mediated chemistry formulation has a disadvantage in that an additional complex purification process is required to separate the reducing agent.

Meanwhile, in the past, in order to improve the performance of hetero-nanoparticles used for the detection of targets in the human body or as drug carriers or the like, the surface of hetero-nanoparticles was coated with a cationic material. In general, materials that exhibit cationic properties while having biocompatibility mainly contain an amine group, and the amine group may cause a problem of binding to surface receptors of macrophages in the human body, thereby causing an inflammatory response.

### PRIOR ART DOCUMENT

### Patent Document

(Patent Document 1) Korean Registered Patent No. 10-1467938

### SUMMARY OF THE INVENTION

The present disclosure provides a nano complex material which can be used in many ways in the biotechnology field by exhibiting excellent light absorbance, magnetic absorbance, storage stability, drug delivery performance, and low cytotoxicity; a drug carrier including the same; a contrast agent including the same; and a method for preparing the nano complex material.

The present disclosure relates to a nano complex material. The nano complex material includes a support including an inorganic material; and ionic particles formed on the support and including a metal having a work function of 6.0 eV or less, and since the ratio of ionic mobilities, which is measured by using a tandem differential mobility analyzer, is adjusted within a specific range, the nano complex material exhibits excellent light absorbance, magnetic absorbance, storage stability, drug delivery performance, and low cytotoxicity.

The present disclosure relates to a drug carrier. Since the drug carrier includes the nano complex material described above, they can be used for simultaneously performing treatment and diagnosis.

The present disclosure relates to a contrast agent. Since the contrast agent includes the nano complex material described above, the contrast agent can provide images with excellent contrast as a contrast agent for MRI-CT dual-mode imaging devices.

The present disclosure relates to a method for preparing a nano complex material. The method sequentially performs a forming step of forming ionic particles including a metal having a work function of 6.0 eV or less; a binding step of binding the ionic particles to droplets which are formed by spraying a solution including a support and a solvent; and a light irradiating step of irradiating the droplets bound with the ionic particles with ultraviolet rays having a wavelength range of 200 nm or less. Therefore, the method is environmentally friendly, and it is easy to design, prepare, and modify a nano complex material which is suitable for the purpose of use, and it is possible to continuously prepare the nano complex material.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a diagram schematically illustrating an exemplary nano complex material of the present disclosure;

FIG. 2 is a graph showing the results of measuring the size distribution of the nano complex material prepared in Example and nanoparticles prepared in Comparative Example 1 and Comparative Example 2;

FIGS. 3 to 5 are transmission electron microscope (TEM) images of the nanoparticles prepared in Comparative Example 1 and Comparative Example 2 and the nano complex material prepared in Example respectively;

FIG. 6 is a set of scanning electron microscope (SEM) images of the nano complex material prepared in Example;

FIG. 7 is a diagram schematically illustrating a process of forming the nano complex material according to Example;

FIG. 8 shows UV-vis absorption spectra of the nano complex material prepared in Example and the nanoparticles prepared in Comparative Example 1 and Comparative Example 2;

FIG. 9 shows the results of measuring the X-ray diffraction (XRD) patterns of the nano complex material prepared in Example and the nanoparticles prepared in Comparative Example 1 and Comparative Example 2;

FIG. 10 is a graph showing the results of measuring the magnetic properties of the nano complex material prepared in Example and the nanomaterial prepared in Comparative Example 2;

FIG. 11 is a graph showing the results of measuring the cytotoxicity of the nano complex material prepared in Example and the nanoparticles prepared in Comparative Examples 1 to 3;

FIG. 12 is a set of images showing the measurement of the cell compatibility of the nano complex material prepared in Example;

FIG. 13 is a graph showing the evaluation of the gene delivery performance of the nano complex materials prepared in Example and Comparative Example 3;

FIG. 14 is a graph showing the measurement of the charge distribution of the nano complex material prepared in Example as obtained using a tandem differential spectrometer;

FIG. 15 is a graph showing the measurement of the Au 4f spectrum of the nano complex materials prepared in Example and Comparative Example 3 as obtained by XPS;

FIG. 16 is a graph of the results of measuring the cell uptake rate of the nano complex material prepared in Example;

FIG. 17 is a schematic diagram of an experiment for determining the photothermal therapy performance of the nano complex material prepared in Example;

FIG. 18 is a graph of the results of measuring the photothermal therapy performance of the nano complex material prepared in Example according to the experimental method of FIG. 17;

FIG. 19 is a graph showing the evaluation of the cell-killing performance of the nano complex material prepared according to the experimental method of FIG. 17; and

FIG. 20 is a graph showing the comparison of degrees of inhibition of production of macrophage inflammatory protein (MIP) of LPS-macrophage by the nano complex material prepared in Example and control groups.

### DETAILED DESCRIPTION OF EXEMPLARY EMBODIMENTS

The present disclosure relates to a nano complex material. Since the nano complex material consists of components each having a different function, it can be used, for example, as a contrast agent for MRI-CT dual-mode imaging devices or a drug carrier by which treatment and diagnosis are simultaneously performed.

An exemplary nano complex material of the present disclosure may have a support including an inorganic material; and ionic particles which are formed on the support and include a metal having a work function of 6.0 eV or less, wherein the charge number (q) of the nano complex material which is determined by Equation 1 below may be in a range of 1 to 50, 1 to 40, 1 to 30, 1 to 20, or 1 to 10.

$$q = \frac{{Z}_{1}}{{Z}_{2}} \qquad [ Equation 1 ]$$

In Equation 1, Z₁ represents the ionic mobility of the nano complex material, which is measured by using a first differential mobility spectrometer of a tandem differential mobility analyzer (TDMA), and Z₂ represents the ionic mobility of the nano complex material, which is measured by using a second differential mobility spectrometer.

The tandem differential mobility analyzer is a device for measuring the mobility of a material having an ionic property. When an electric field is applied inside the tandem differential mobility analyzer, the material having an ionic property has mobility and sequentially passes through the first and second differential mobility spectrometers. For example, it may indicate that a nano complex material having a greater charge number according to Equation 1 is a material that passes through the first and second differential spectrometers at a higher velocity than a nano complex material having a lower charge number.

In one example, the charge number of Equation 1 may be the surface charge amount of the nano complex material. Specifically, the fact that the charge number according to Equation 1 is large means that the surface charge amount of the nano complex material is large, and in other words, it means that the nano complex material exhibits sufficient ionicity. On the other hand, when the surface charge amount of the nano complex material is small, the nano complex material may not exhibit sufficient ionicity. In this case, the nano complex material may have a charge number of less than 1, which is determined by Equation 1.

The nano complex material according to the present disclosure exhibits excellent light absorbance, magnetic absorbance, storage stability, drug delivery performance, and low cytotoxicity because the charge number (q) which is determined by Equation 1 is adjusted within a specific range.

The term “nano” as used herein may refer to a size in nanometer (nm) units, and for example, it may refer to a size of 1 to 1,000 nm, but is not limited thereto. In addition, the term “nano complex material” as used herein may refer to a material which has an average diameter in nanometer (nm) units and is prepared by combining two or more types of different materials and thus has excellent physical or chemical functions different from those of the original materials. For example, the nano complex material may refer to a material having an average diameter of 1 to 1,000 nm, but is not limited thereto.

In addition, the term “ionic particle” as used herein may refer to a particle whose surface charge is expressed as a cation or an anion, and for example, the term may refer to a particle whose surface charge is expressed as a cation in the present disclosure.

Hereinafter, the nano complex material of the present disclosure and the method for manufacturing the nano complex material will be described with reference to the accompanying drawings, but the accompanying drawings are illustrative, and the scope of the present disclosure is not limited to the accompanying drawings.

The exemplary nano complex material of the present disclosure has a structure in which ionic particles are randomly disposed on the surface of a support. The phrase, “nano complex material has (or substantially consist of) support and ionic particles” as used herein means that the nano complex material does not substantially include other components besides the ionic particles and the support. In addition, the fact that the nano complex material does not substantially include the other components means that the nano complex material may include other components within a content range in which the physical or chemical properties of the nano complex material are not modified by the other components. For example, the content at which the properties are not modified may be 1 part by weight or less, 0.1 part by weight or less, 0.01 part by weight or less, or 0.001 part by weight or less, based on 100 parts by weight of the nano complex material.

In one example, the nano complex material having the support and ionic particles may not include an organic polymer. The organic polymer may be, for example, a cationic material which is described below.

In one example, the nano complex material of the present disclosure may be hetero-particles in which materials constituting a support and ionic particles may be different from each other. The term “hetero-particle” as used herein refers to a particle including two or more types of different materials in the particle at similar masses, and for example, a mass ratio may be 1:1, 1:1.5, or 1:2.

In one embodiment, the inorganic material may be a magnetic material, silica, or alumina. The nano complex material of the present disclosure may be used as a biomaterial having various functions by including a support including the inorganic material, and ionic particles. For example, a nano complex material, which has a support including a magnetic material, and ionic particles, may be used as a contrast agent for an MRI-CT dual-mode imaging device which is described below. A nano complex material including a support including silica, and ionic particles may be used as a drug carrier by which treatment and diagnosis can be simultaneously performed. In addition, a nano complex particle including a support including alumina, and ionic particles may be used as a photothermal therapy agent.

In one example, the magnetic material may be a ferromagnetic material, a paramagnetic material, or a diamagnetic material.

The term “magnetic material” as used herein refers to a material magnetized in a magnetic field, and the term “ferromagnetic material” refers to a material which becomes strongly magnetized in the direction of an applied external magnetic field and exhibits magnetic hysteresis by leaving residual magnetization even when the magnetic field is removed. In addition, the term “paramagnetic material” as used herein refers to a material which has a magnetic property by becoming magnetized in the direction of an applied external magnetic field and loses a magnetic property when the external magnetic field is removed. The term “diamagnetic material” refers to a magnetic material in which magnetization occurs in the opposite direction of an external magnetic field.

In one example, the ferromagnetic material may be a metal such as iron, cobalt, nickel, or the like, or an alloy, sulfide, or oxide of the metal, and for example, the ferromagnetic material may be iron oxide (Fe₃O₄), but is not limited thereto. In particular, since iron oxide is a material which has strong magnetic absorption and is relatively not harmful to the human body, the nano complex material including the same may be appropriately used as an MRI contrast agent which can be injected into the blood of the human body.

The paramagnetic material may be at least one metal selected from the group consisting of platinum (Pt), tin (Sn), tungsten (W), molybdenum (Mo), aluminum (Al), manganese (Mn), palladium (Pd), rhodium (Rh), ruthenium (Ru), zirconium (Zr), europium (Eu), and dysprosium (Dy).

The diamagnetic material may be at least one metal selected from the group consisting of gold (Au), silver (Ag), bismuth (Bi), and tantalum (Ta).

In one example, the metal may have a work function of 6.0 eV or less, for example, a work function of 5.6 eV or less or 4.9 eV or less, but it is not particularly limited thereto. For metals that have a work function within the above range, electrons on a metal surface are ejected due to the emission of light having a photon energy exceeding 6.0 eV, for example, light having a short wavelength of 200 nm or less such as ultraviolet rays and the like, however, the ionic particles of the present disclosure include the metals from which electrons have been previously ejected. Accordingly, the surface of the nano complex material including the ionic particles may be positively charged.

In one example, the metal having a work function of 6.0 eV or less may be at least one selected from the group consisting of barium, silver, cadmium, aluminum, beryllium, cerium, cesium, cobalt, chromium, iron, gallium, gadolinium, hafnium, mercury, indium, magnesium, manganese, molybdenum, lead, niobium, neodymium, rubidium, rhenium, rhodium, ruthenium, scandium, tin, strontium, tantalum, terbium, tellurium, thorium, titanium, uranium, vanadium, yttrium, thallium, ytterbium, zinc, palladium, iridium, platinum, gold, and zirconium.

Specifically, the metal having a work function of 6.0 eV or less may be gold (Au). Since gold (Au) is a metal which has strong X-ray absorption and also is relatively not harmful to the human body, the nano complex including the same may be appropriately used as a CT contrast agent, a photothermal therapy agent, or the like that is injected into the blood in the human body.

In one embodiment, the metal may be an agglomerate, and the average particle diameter of the aggregate may be in a range of 2 nm to 40 nm, 2 nm to 30 nm, or 2 nm to 25 nm.

In one example, the surface of the ionic particle may be positively charged. In the case of conventional nanoparticles used as biomaterials, they were coated with a cationic material to improve biocompatibility and functionality. However, most cationic materials have an amine group, and this amine group may cause a problem of binding to the surface receptors of macrophages in the human body, thereby causing an inflammatory response. On the other hand, since the nano complex material of the present disclosure has a positive charge on its own, a process of coating with a cationic material is not required, and therefore, an inflammatory response caused by the amine group of a cationic material can be minimized.

In one example, the nano complex material may have a peak change rate (ΔP) in a range of 0.1 to 20 eV, 0.1 to 15 eV, or 0.1 to 10 eV, which is determined by Equation 2 below, during X-ray photoelectron spectroscopy analysis.

ΔP=|Pₘ−Pᵢ| [Equation 2]

In Equation 2, Pₘ represents an XPS peak of a metal having a work function of 6.0 eV or less, and Pᵢ represents an XPS peak of the ionic particles comprising the metal having a work function of 6.0 eV or less. The analysis was carried out using an X-ray photoelectron spectroscope (Kratos Axis HIS). The XPS peak may denote binding energy.

In one embodiment, the nano complex material may have a support including iron oxide and ionic particles including gold. In this case, the nano complex material may exhibit excellent X-ray absorption and magnetic absorbance. In general, magnetic resonance imaging (MRI) or computed tomography (CT) imaging records the difference in brightness between a light emitting portion and a light absorbing portion. That is, the fact that X-ray absorption or magnetic absorbance is excellent means that image signals of the light absorbing portion are strong. Therefore, when the nano complex material having a support including iron oxide, and ionic particles including gold is applied as a contrast agent in a CT, MRI, or MRI-CT dual-mode imaging device, it can induce strong image signals to provide images with an excellent contrast. In addition, since iron oxide and gold have low cytotoxicity in the human body, the nano complex material may exhibit low cytotoxicity.

In one embodiment, the nano complex material having a support including iron oxide, and ionic particles including gold may have an effective peak within a range of 83.8 eV to 87.5 eV during X-ray photoelectron spectroscopy analysis. An effective peak within the above range may be produced due to a metal that has been induced to exhibit cationicity. For example, the effective peak may be a peak produced due to a gold cation (Au^{δ+}).

The present disclosure relates to a drug carrier including the nano complex material and a drug supported in the nano complex material. The drug carrier of the present disclosure may include the nano complex material described above, and therefore, contents overlapping with those described for the nano complex material will be omitted. An exemplary drug carrier according to the present disclosure has excellent storage stability, drug (including genes) delivery performance, and low cytotoxicity by including the nano complex material described above. In particular, the drug carrier has an excellent binding force with a negatively charged gene or drug due to the nano complex material whose surface is positively charged and exhibits an excellent anti-inflammatory property at the same time.

In one example, the drug may be at least one selected from the group consisting of an antifungal agent, an antibacterial agent, an antimicrobial agent, an antioxidant, a coolant, a soothing agent, a wound-healing agent, an anti-inflammatory agent, an anti-aging agent, an anti-wrinkle agent, a skin-lightening agent, a bleaching agent, a light-absorbing agent, a scattering agent, a skin-bleaching agent, a dye, a coloring agent, a deodorant, and a fragrance, but is not limited thereto.

The present disclosure relates to a contrast agent including the nano complex material. The contrast agent of the present disclosure may include the nano complex material described above, and therefore, contents overlapping with those described for the nano complex material will be omitted. The term “contrast agent” (contrast medium) mentioned above refers to a functional chemical that is introduced into the stomach, intestines, blood vessels, cerebrospinal fluid, articular cavities, and the like, in order to make it easy to distinguish tissues or blood vessels during radiological examinations using equipment such as an MRI device and a CT device.

An exemplary contrast agent of the present disclosure has excellent light absorbance, magnetic absorbance, cell absorption, and low cytotoxicity by including the nano complex material described above, and can be used in many ways in the human body. In particular, in the case of a contrast agent including the nano complex material which has a support including iron oxide, and ionic particles including gold, it has excellent cell absorption, light absorbance, and magnetic absorbance when applied in an MRI-CT dual-mode imaging device, and as a result, it can induce strong imaging signals to realize images having an excellent contrast. In addition, as described above, since the surface of the nano complex material of the present disclosure is positively charged on its own without being coated with a cationic material, the contrast agent including the same may effectively suppress inflammatory responses caused by a cationic material in the human body.

In addition, the present disclosure relates to a method for preparing the nano complex material. According to an exemplary method for preparing the nano complex material according to the present disclosure, the nano complex material may be continuously prepared through a simple and environmentally friendly process.

FIG. 1 is a diagram schematically illustrating an exemplary method for preparing the nano complex material of the present disclosure. As shown in FIG. 1, the exemplary method for preparing the nano complex material of the present disclosure includes a forming step, a binding step, and a light irradiating step.

The forming step is a step of forming metal particles having a work function of 6.0 eV or less. In one example, the forming step may be a step of generating metal particles from an electrode surface by applying a spark discharge voltage to a conductive rod. The spark discharge voltage may be appropriately adjusted by an electrode interval, an applied current, a capacitance, and the like.

In the forming step, for example, when the interval between electrodes is 1 mm, a high temperature of about 5,000° C. may occur when a voltage of 2.5 to 3.5 kV is applied. After a metal constituting the electrode is sublimated due to the high temperature, metal particles can be formed while the sublimated metal is rapidly condensed at room temperature due to deviating from the interval between electrodes at which the high temperature is generated. The metal particles may be formed as ionic particles through a light irradiating step described below.

In one example, the method for preparing the nano complex material of the present disclosure may further include a gas supplying step of supplying an inert gas or nitrogen between the electrodes. The metal particles formed in the forming step may move to the binding step and the light irradiating step described below along the flow of the inert gas or nitrogen supplied in the gas supplying step.

The binding step is a step of binding the metal particles to droplets which are formed by spraying a solution including a support and a solvent.

In one example, the binding step may include a mixing step of mixing the support and the solvent. A solution prepared in the mixing step may be sprayed in droplets by a spraying device and bind with metal particles that have moved along the flow of an inert gas or nitrogen.

The spraying device is not particularly limited as long as it is, for example, a device equipped with a nozzle which can spray a solution to form droplets. The diameter of the nozzle is not particularly limited, but may be, for example, 0.1 to 1.0 mm.

In the mixing step, the support and solvent may be stirred at a rate of 200 to 4,000 rpm. In one example, the volume fraction of the support in the solution prepared in the mixing step may be 0.005 to 15 parts by volume, 0.005 to 10 parts by volume, or 0.005 to 5 parts by volume, based on 100 parts by volume of the entire solution.

The solvent is a component which is mixed with a support to spray the support in the form of droplets and is not particularly limited as long as it is highly compatible with the support, and for example, the solvent may be ethanol, methanol, propanol, dichloromethane, distilled water, hexane, or dimethyl sulfoxide (DMSO).

The light irradiating step is a step of irradiating droplets bound with the metal particles with ultraviolet rays having a wavelength range of 200 nm or less. In the light irradiating step, ultraviolet rays having a wavelength range of 200 nm or less, for example, 180 nm or less, or 160 nm or less may be emitted to droplets bound with metal particles that have been delivered by an inert gas or nitrogen. Electrons present on the surface of metal particles may be ejected by the light irradiation. In the present disclosure, ionic particles may refer to metal particles whose surface has been induced to have a positive charge due to the ejection of the electrons.

For a ultraviolet irradiating device, a device that can emit light having a photon energy of 6.0 eV or more (for example, light having a short wavelength of 200 nm or less) can be used without limitation. For example, a known light source such as a high-pressure mercury lamp, an ultra-high-pressure mercury lamp, a halogen lamp, a black light lamp, a microwave-excited mercury lamp, various lasers, X-rays, or the like may be used. In another example, the light irradiating step may be performed through emission of soft X-rays along the flow of an inert gas at room temperature.

The exemplary method for preparing the nano complex material of the present disclosure may further include an extracting step after the light irradiating step.

In one example, the extracting step may be a step of extracting a solvent via a solvent extraction method or drying. The nano complex material in which the solvent has been extracted may be present in a powder form which exhibits excellent storage stability.

In one example, the drying may be carried out in a temperature range of, for example, 60° C. to 250° C., 70° C. to 200° C., or 80° C. to 170° C. When the drying temperature exceeds 200° C., the support may be deformed or decomposed, and when the drying temperature is lower than 60° C., the solvent of interest may not be sufficiently removed.

In addition, the method for preparing the nano complex material of the present disclosure may further include a collecting step of collecting the nano complex material in a powder form with a substrate or a filter.

As described above, the method for preparing the nano complex material of the present disclosure may be an aerosol process in which the forming step, the binding step, and the light irradiating step are carried out under the flow of an inert gas. The term “aerosol” mentioned above refers to solid or liquid nanoparticles suspended in the atmosphere. That is, the method for preparing the nano complex material of the present disclosure may be an aerosol process because nano-sized materials react under the flow of an inert gas or nitrogen.

Hereinafter, the contents disclosed above will be described in detail with reference to Example and Comparative Examples. However, the scope of the present disclosure is not limited by the following description.

### Preparation of Ionic Particles and Nano Complex Material

### Example—Au*@Fe₃O₄

According to the method of FIG. 1, the nano complex material was prepared.

Specifically, as shown in FIG. 1, nitrogen gas was passed between a pair of electrodes made of a gold (Au) rod (diameter 3 mm, length 100 mm, Nilaco, Japan). In this case, the interval between the pair of electrodes was set to 1 mm Spark ablation was generated by applying power to the electrodes to prepare gold nanoparticles. Specifically, for the spark ablation, a power source under conditions of a resistance of 0.5 MΩ, a capacitance of 1.0 nF, a current of 1.2 mA, a voltage of 2.6 kV, and a frequency of 380 Hz was applied to the electrodes.

The nitrogen gas (purity exceeding 99.99%) including the generated gold (Au) nanoparticles was flowed toward the spray device and used as an operational gas for spraying the solution including a support.

In order to prepare a solution including a support, first, 0.074 g of FeCl₃.6H₂O (Sigma-Aldrich, US) and 0.026 g of FeCl₂. 6H₂O (Sigma-Aldrich, US) were mixed with 30 mL of ethanol to prepare precursor solution 1. Then, 5 mL of ammonia (28-30%) was mixed with 25 mL of ethanol to prepare precursor solution 2.

The precursor solution 1 and precursor solution 2 were introduced into a container at a rate of 6 mL/min and 4 mL/min, respectively, by using a peristaltic pump (323 Du/MC4, Watson-Marlow Bredel Pump, US). Then, an ultrasonic probe (VCX 750, 13 mm titanium alloy horn, 20 kHz, Sonics & Materials Inc., US) was immersed in the container including the mixed solution to prepare a solution including a support. The probe was operated at a power density of 10 W/mL, and the area of the bottom part of the probe was 1.3 cm².

The solution was sprayed in the form of droplets using a spraying device equipped with an injection nozzle with an outlet having a diameter of 0.3 mm Gold-iron oxide (Au @Fe₃O₄) hybrid droplets were prepared by combining gold nanoparticles that have been introduced through the nitrogen gas flow and the sprayed droplets.

The gold-iron oxide (Au@Fe₃O₄) hybrid droplets were passed through the inside of a tubular flow reactor. Specifically, the tubular flow reactor is a device filled with carbon and silica gel inside and provided with a light source which emits light having a wavelength of 185 nm with an intensity of 0.14 J/m²s². The gold-iron oxide (Au@Fe₃O₄) hybrid droplets were ionized as electrons on the surface of the gold nanoparticles were ejected by the light emitted from the light source, and the solvent in the hybrid droplets was extracted by the carbon and the silica gel, and thereby an ionized gold-iron oxide (Au@Fe₃O₄) nano complex material in a powder form was prepared. Then, the ionized gold-iron oxide nano complex material (Au@Fe₃O₄) was collected on a glass substrate (7059, Corning, US).

The ionized gold-iron oxide nano complex material (Au@Fe₃O₄) had a charge number (q) of 1.44 as determined according to Equation 1 described above.

### Comparative Example 1—Au

Gold nanoparticles (Au) were prepared in the same manner as in Example except that a step of spraying the support solution and the light irradiating step were not carried out.

### Comparative Example 2—Fe₃O₄

Iron oxide nanoparticles (Fe₃O₄) were prepared in the same manner as in Example except that a step of forming gold nanoparticles and the light irradiating step were not carried out.

### Comparative Example 3—Au@Fe₃O₄

The non-ionized gold-iron oxide nano complex material (Au@Fe₃O₄) was prepared in the same manner as in Example except that the light irradiating step was not carried out.

### Experimental Example—Property Evaluation of nano complex material

<Evaluation of Physical Properties of Nano Complex Material>

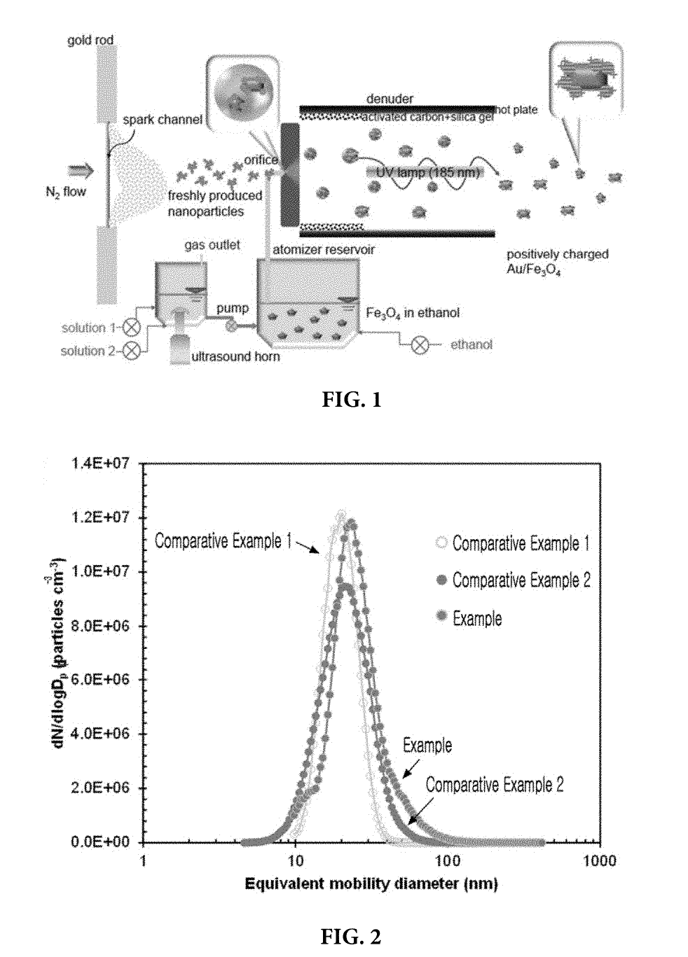

FIG. 2 is a graph showing the results of measurement of the size distribution of the nano complex material prepared in Example, and the nanoparticles prepared in Comparative Example 1 and Comparative Example 2. The measurement was carried out using a scanning mobility particle sizer (SMPS) (3936, TSI, USA), and the average particle diameters, standard deviations (SD), and number concentrations were measured. The measurement results are shown in Table 1 below.

TABLE 1

| | Average particle | Standard | Number |
|---|---|---|---|
| | diameter | deviation | concentration |
| Classification | (nm) | (—) | (cm⁻³) |
| Example (Au*@Fe₃O₄) | 24.7 | 1.49 | 4.10 × 10⁶ |
| Comparative | 19.7 | 1.28 | 3.45 × 10⁶ |
| Example 1 (Au) | | | |
| Comparative | 20.2 | 1.46 | 3.22 × 10⁶ |
| Example 2 (Fe₃O₄) | | | |

As shown in Table 1 above and FIG. 2, the nano complex material (Au*@Fe₃O₄) of Example consisted of gold and iron oxide nanoparticles having different size distributions, but the measurement result showed a uniform distribution. Further, referring to FIG. 2, the size distribution of the nano complex material (Au*@Fe₃O₄) of Example was similar to that of the gold (Au) nanoparticles, rather than that of the iron oxide (Fe₃O₄) nanoparticles. This suggests that the gold (Au) nanoparticles constituting the nano complex material (Au*@Fe₃O₄) were quantitatively and completely bound on the iron oxide (Fe₃O₄) nanoparticles (support).

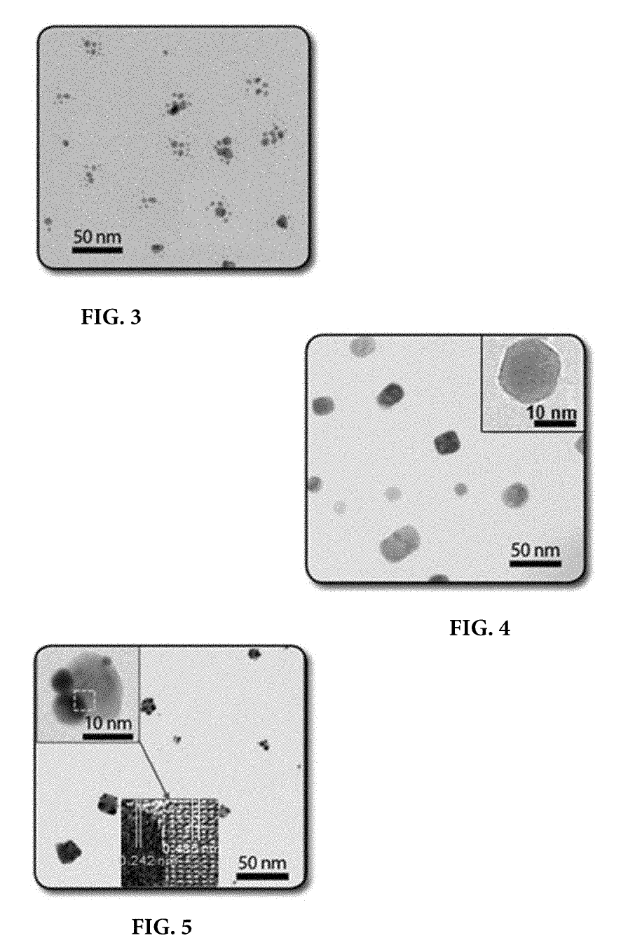

FIGS. 3 to 5 are transmission electron microscope (TEM) images of the nano complex material prepared in Example and the nanoparticles prepared in Comparative Examples 1 and 2. FIG. 3 is a TEM image of the gold nanoparticles prepared in Comparative Example 1, FIG. 4 is a TEM image of the iron oxide nanoparticles prepared in Comparative Example 2, and FIG. 5 is a TEM image of the nano complex material (Au*@Fe₃O₄) prepared in Example.

From FIGS. 3 to 5, the process of forming the nano complex material in Example can be understood. This is because the nano complex material of Example was formed by quantitatively combining the nanoparticles prepared in Comparative Examples 1 and 2. Specifically, the images in FIGS. 3 to 5 were obtained by photographing samples in an aerosol state. The above samples were obtained by using an aerosol collector (NPC-10, HCT, Korea) in the preparation processes of Example and Comparative Examples. In addition, the above images were obtained using a transmission electron microscope (TEM, JEM-3010, JEOL, Japan).

Referring to FIG. 3, the gold nanoparticles were agglomerates having an average diameter of about 3 nm, and this means that gold nanoparticles formed near a spark collided with each other and were held together.

Referring to FIG. 4, it was observed that the iron oxide (Fe₃O₄) nanoparticles prepared according to the present disclosure had a square shape with a size of about 20 nm (see FIG. 4). Although not shown, iron oxide was observed as spherical when Fe(CO)₅ was used as an Fe precursor.

Referring to FIG. 5, a dark region of particles and a bright region of particles having a lattice pattern (111) therein were observed in the nano complex material (Au*@Fe₃O₄) prepared in Example. The lattice pattern in the dark region was due to the face-centered cubic lattice (0.242 nm) of gold nanoparticles. In addition, the lattice pattern in the bright region was caused by the iron oxide nanoparticles, and the interval of the lattice was about 0.485 nm.

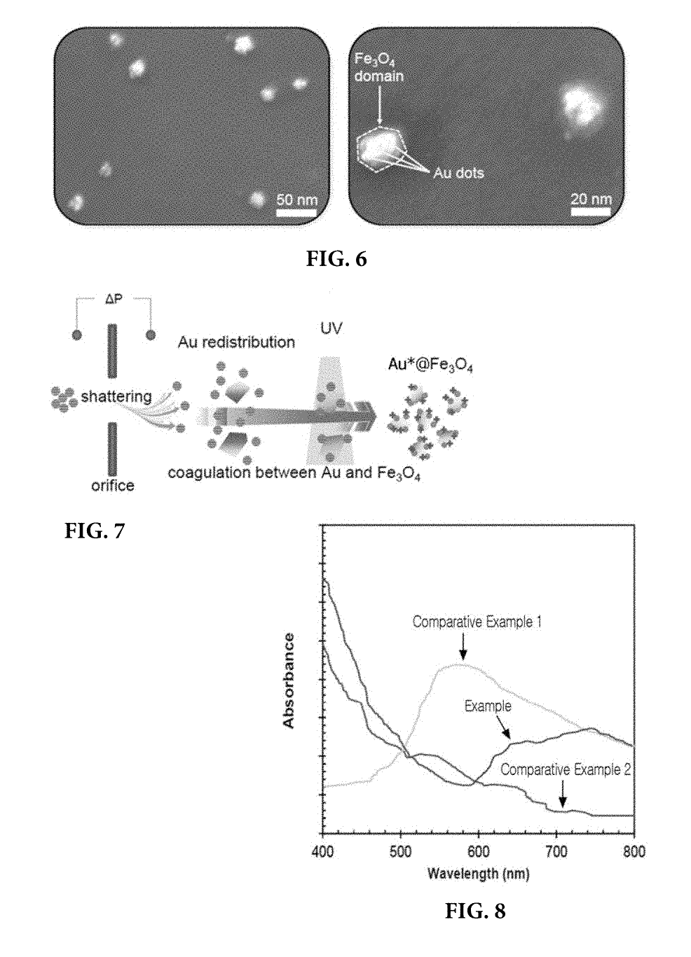

FIG. 6 is a set of scanning electron microscope (SEM) images of the nano complex material (Au*@Fe₃O₄) prepared in Example. The above images were obtained using a scanning electron microscope (SEM, NOVA NanoSEM, FEI, USA).

From FIGS. 3 to 6, it was confirmed that the nano complex material (hetero-nanoparticles) can be prepared even by utilizing aerosol processes that are different from conventional wet chemical formulation.

In addition, referring to FIG. 5, it can be confirmed that agglomerates of gold (FIG. 3) were randomly located in the form of primary particles on the surface of iron oxide nanoparticles due to mechanical restructuring. In this case, the diameter (D_{P}) of the primary particles was calculated through Equation 3 below.

$${D}_{p} = α \sqrt{\frac{{D}_{pa} H}{6 πΔ P {Θ}^{2}}} \qquad [ Equation 3 ]$$

In Equation 3, Dₚₐ is the diameter of a restructured particle, a is the proportionality constant, H is the Hamaker constant, ΔP is the pressure difference, and θ is the cohesive strength parameter.

FIG. 7 is a diagram schematically illustrating the process of forming the nano complex material according to Example.

Referring to FIG. 7, agglomerates of gold (Au) nanoparticles generated from a spark discharge were restructured in the form of primary particles due to various physical conditions (pressure, density, and velocity) in the course of being transferred toward the spraying device. The diameter of the restructured primary particle was about 3 nm or less. The gold nanoparticles were randomly bonded, in the form of primary particles, on the surface of the support including the iron oxide nanoparticles, and the gold-iron oxide nano complex material (Au*@Fe₃O₄) ionized by light irradiation was formed.

The formation of the gold-iron oxide nano complex material (Au*@Fe₃O₄) was also proven from UV-vis absorption spectra.

FIG. 8 shows the UV-vis absorption spectra of the nano complex material prepared in Example, and the nanoparticles prepared in Comparative Example 1 and Comparative Example 2. The spectra were obtained using a UV-vis absorption spectroscope (UV-vis Absorption Spectroscope, 330, Perkin-Elmer, US).

Referring to FIG. 8, the gold nanoparticle of Comparative Example 1 produces an absorption peak at about 525 nm. The spectrum of the nano complex material (Au*@Fe₃O₄) prepared in Example is displayed in a form in which the spectrum of the iron oxide (Fe₃O₄) nanoparticles of Comparative Example 2 has shifted to the near infrared (NIR) region. This suggests that gold (Au) nanoparticles were well bound onto the iron oxide (Fe₃O₄) support of the nano complex material (Au*@Fe₃O₄) prepared in Example.

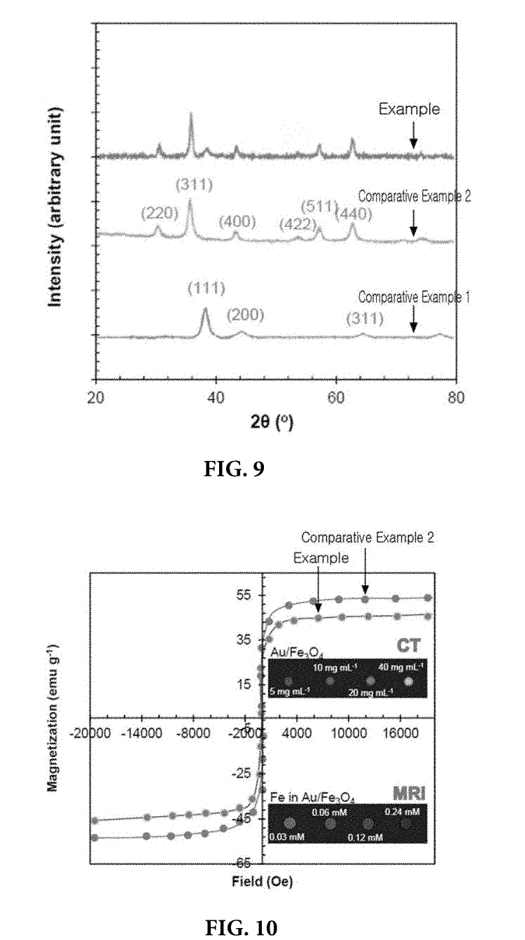

FIG. 9 is a graph showing the results of measuring the X-ray diffraction (XRD) pattern of the nano complex material prepared in Example, and the nanoparticles prepared in Comparative Example 1 and Comparative Example 2.

Referring to FIG. 9, six peaks at 30.4° (220), 35.4° (311), 43.2° (400), 53.4° (422), 57.2° (511) and 62.7° (440) represent the intrinsic peaks of the iron oxide (Fe₃O₄) nanoparticles prepared in Comparative Example 2. On the other hand, the nano complex material (Au*@Fe₃O₄) prepared in Example produces the six peaks and, additionally, a 38.1° (111) peak (peak due to the gold nanoparticle). This suggests that gold (Au) nanoparticles were well bound onto the iron oxide (Fe₃O₄) support of the nano complex material (Au*@Fe₃O₄) prepared in Example.

<Performance Evaluation of Contrast Agent for Magnetic Resonance Imaging (MRI) and In Vitro Computed Tomography (CT)>

Samples of aqueous dispersions including the nano complex material (Au*@Fe₃O₄) prepared in Example at varying mass concentrations were prepared. The contrast performance of the samples was evaluated using a 9.4T MRI device for small animals (Bruker). Specifically, the performance was evaluated via T₂-weighted imaging. T₂-weighted imaging was carried out under device conditions of a TE of 4 ms, a slice thickness of 0.5 mm, a field of view of 3 cm×3 cm, and an inversion recovery sequence with a matrix size of 128×128. The contrast performance of each sample was evaluated based on Hounsfield units.

Samples including the nano complex material (Au*@Fe₃O₄) prepared in Example at varying mass concentrations were prepared in 2.0 mL Eppendorf tubes. Then, the prepared samples were placed into a self-designed scanning holder. CT scans were performed using a GE Light Speed VCT imaging system (GE Medical Systems) operating at 100 kV and 80 mA, with a slice thickness of 0.625 mm. The contrast performance of each sample was evaluated based on Hounsfield units.

Using a vibrating sample magnetometer (7404, Lake Shore Cryotronics, US) at a temperature of 300 K, the magnetic properties of the nano complex material prepared in Example were measured, and the results are shown in FIG. 10.

FIG. 10 is a graph showing the results of measuring the magnetic properties of the nano complex material prepared in Example and the nanomaterial prepared in Comparative Example 2.

Referring to FIG. 10, the nano complex material (Au*@Fe₃O₄) prepared in Example shows a lower saturation magnetization than that of the iron oxide (Fe₃O₄) nanoparticles of Comparative Example 2. This is because the gold nanoparticles bound to the iron oxide nanoparticles contribute to diamagnetization. Although the nano complex material (Au*@Fe₃O₄) of Example shows a lower magnetization value than the iron oxide (Fe₃O₄) nanoparticles of Comparative Example 2, the magnetization value is sufficient for the nano complex material to be used as a contrast agent for MR and/or CT devices.

Specifically, in order to evaluate the functionality as a contrast agent of the nano complex material (Au*@Fe₃O₄) prepared in Example, samples for MR and CT were prepared. The samples were prepared by suspending the nano complex material (Au*@Fe₃O₄) in a gas phase in water at varying mass concentrations (CT samples were adjusted to a mass concentration of mg/mL, and MR samples were adjusted to a mass concentration of mM). Referring to the illustration in FIG. 10, in the CT image, the signal intensity increased as the mass concentration of the nano complex material (Au*@Fe₃O₄) increased. In addition, in the MR image, the signal intensity increased as the mass concentration of the nano complex material (Au*@Fe₃O₄) decreased. From the above results, it was found that the nano complex material (Au*@Fe₃O₄) prepared in Example sensitively interacts in the CT and MR images. This suggests that the nano complex material (Au*@Fe₃O₄) prepared in Example can be used as a contrast agent suitable for MRI-CT dual-mode imaging devices.

In addition, in order to evaluate the storage stability of the nano complex material (Au*@Fe₃O₄) prepared in Example, dynamic light scattering (DLS) (Nano ZS90, Malvern Instruments, Worcestershire, UK) measurement was carried out. Prior to evaluating storage stability, samples were prepared by collecting the nano complex material in an aerosol state on a glass plate. All evaluated samples showed a deviation in a hydrodynamic diameter of 6.1% or less, with no significant change during a storage period of 1 to 14 days. From this fact, it was found that the nano complex material of Example has excellent storage stability.

<Cell Viability Evaluation>

According to an analysis with MTS (3-(4,5-dimethyl-thiazol-2-yl)-5-(3-carboxymethoxyphenyl)-2-(4-sulfophenyl)-2H-tetrazolium), cytotoxicity of nano complex material (Au*@Fe₃O₄) prepared in Example was evaluated in HeLa cells (HEK 293). HeLa cells (HEK 293) were cultured in 200 mL of Dulbecco's Modified Eagle's Medium (DMEM, Carlsbad, USA) supplemented with 10% fetal bovine serum (FBS) at 37° C., 5% CO₂, and 95% relative humidity. Then, the cultured cells were applied to a 96-well microtiter plate (Nunc, Germany) at a density of 1×10⁵ cells/well. After 24 hours, the Eagle's medium was replaced with a serum-supplemented culture medium containing chitosan (1 mg/mL) and the cells were cultured for another 24 hours. Thereafter, 30 μL of an MTS reagent was added to each well and the cells were further cultured for 2 hours. Afterwards, absorbance was measured at a wavelength of 490 nm using a microplate reader (Spectra Plus, TECAN, Switzerland). Cell viability (%) was compared with the medium to which cells untreated with the nano complex material (control group) were applied and calculated by Equation 4 below.

[A]ₜₑₛₜ=[A]_{control}×100% [Equation 4]

In Equation 4, the [A]ₜₑₛₜ is the absorbance of a well with the nano complex material, and [A]_{control} is the absorbance of a comparison well. All experiments were carried out 3 times, and the results were expressed as mean and standard deviation. For statistical analysis, the Student's T-Test was used. Differences were considered to be significant at p<0.05.

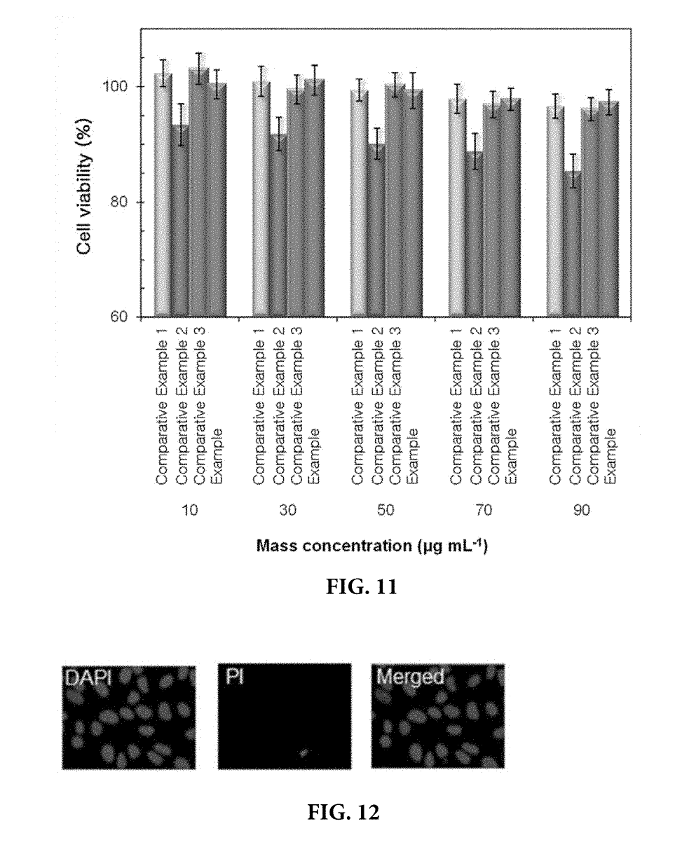

FIG. 11 is a graph of the results of measuring the cytotoxicity of the nano complex material prepared in Example and the nanoparticles prepared in Comparative Examples 1 to 3.

Specifically, FIG. 11 is a graph comparing the results of measuring the cytotoxicity of the nano complex material of Example and the nanoparticles of Comparative Examples against HeLa Cells using MTS analysis. As shown in FIG. 11, the nano complex materials prepared in Example and Comparative Example 3 showed a cell viability of 96% or more in a concentration range of 10 to 90 μg/mL. On the other hand, the gold nanoparticles of Comparative Example 1 showed a cell viability of 97% or more, and the iron oxide nanoparticles of Comparative Example 2 showed a cell viability of 85% or more.

FIG. 12 is a set of images obtained by measuring the cell compatibility of the nano complex material prepared in Example. Specifically, in order to evaluate the biocompatibility of the nano complex material (Au*@Fe₃O₄), 4′-6-diamino-2-phenylindole (DAPI, Sigma-Aldrich, US) and propidium iodide (PI, BD Sciences, US) fluorescent dyes were used. PI (Red-488 nm) can pass through the membrane of dead cells, whereas DAPI (Blue-350 nm) stains both living and dead cells. Fluorescence expression was measured using a fluorescence analyzer (IX 71, Olympus, US), and from FIG. 12, it was confirmed that the nano complex material (Au*@Fe₃O₄) of Example was biocompatible. These results support the MTS analysis results described above. In particular, the nano complex material (Au*@Fe₃O₄) of Example does not show a significant difference from the gold (Au) nanoparticles in terms of cytotoxicity, and this suggests that the gold (Au) nanoparticles can reduce the relatively high cytotoxicity of the iron oxide (Fe₃O₄) nanoparticles. In particular, by binding to the iron oxide nanoparticles, the gold nanoparticles can inhibit intracellular enzymatic degradation, which can be caused by the catalytic oxidative stress of iron oxide nanoparticles.

<Evaluation of Gene Delivery (Transfection) Performance>

24 hours before transfection, HeLa cells (HEK 293) were cultured in 1 mL of Dulbecco's Modified Eagle's Medium (DMEM, Carlsbad, USA) supplemented with 10% fetal bovine serum (FBS) at 37° C., 5% CO₂ and 95% relative humidity. Then, the cultured HeLa cells (HEK 293) were applied to a 24-well plate at a density of 1×10⁶ cells/well. Afterwards, the medium was replaced with Dulbecco's Modified Eagle's Medium without fetal bovine serum (FBS), and a transfection complex (the nano complex material of Example to which a fluorescent protein was attached) was applied to the medium. After replacement with a fresh medium, the transfection complex and the cells were cultured together at 37° C. for 24 hours. The medium was aspirated and washed with phosphate-buffered saline. Following the addition of trypsin, the cells that were activated by fluorescence were measured to assess transfection. Expression of the fluorescent protein was observed using a luminometer (9100-102, Turner Biosystems, US). The degree of the final expression of the fluorescent protein was expressed as relative light-emitting unit (RLU)/mg.

All experiments were carried out 3 times, and the results were expressed as mean and standard deviation. Statistical analysis was performed using the Student's T-Test. Differences were considered to be significant at p<0.05.

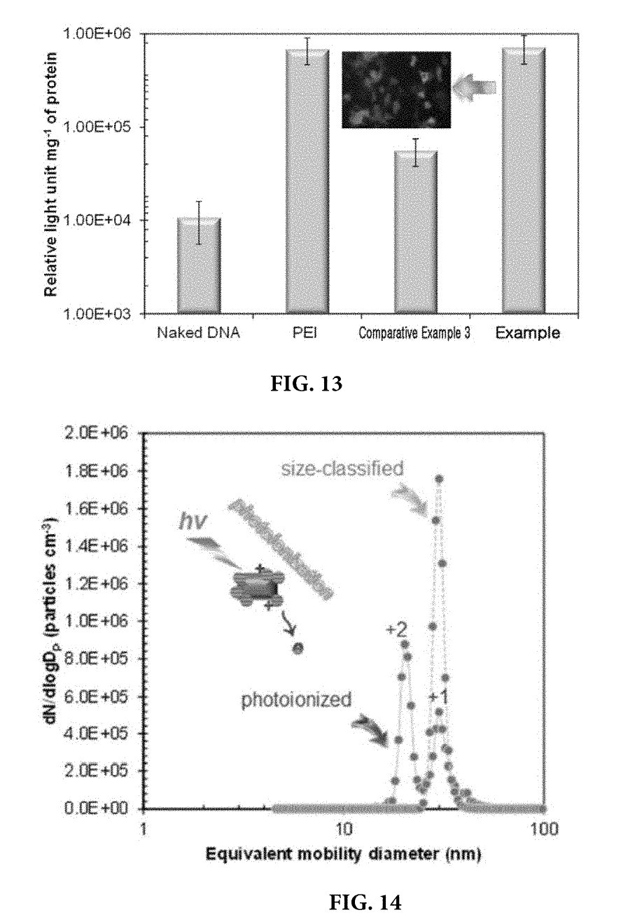

FIG. 13 is a graph showing the evaluation of the gene delivery performance of the nano complex materials prepared in Example and Comparative Example 3. Specifically, a plasmid DNA was mixed with each of the nano complex materials prepared in Example and Comparative Example 3 to prepare samples in which DNA and the nanomaterials were bound. In addition, necked DNA and PEI were used as control groups. The control groups and the prepared samples were injected into HeLa cells (HEK 293). Referring to FIG. 13, the nano complex material (Au*@Fe₃O₄) prepared in Example showed more excellent gene delivery performance than that of the nano complex material (Au@Fe₃O₄) prepared in Comparative Example 3 and the DNA (control group).

The illustration of FIG. 13 is an image showing the expression of a green fluorescent protein (EGFP) of a sample in which DNA is bound to the nano complex material (Au*@Fe₃O₄) prepared in Example in the cell. From the illustration, it can be confirmed that the nano complex material prepared in Example has excellent gene delivery performance.

The improved performance of the ionized nano complex material (Au*@Fe₃O₄) compared to the nano complex material of Comparative Example 3 (Au@Fe₃O₄) may be due to the positively charged surface of the ionized nano complex material.

<Measurement of Surface Charge of Nano Complex Material>

A tandem differential mobility analyzer (TDMA) consisting of first and second differential mobility spectrometers (NDMA 1 and 2, 3085, TSI, US) and a condensation particle counter (CPC, 3776, TSI, US) were used to measure the surface charge of the nano complex material (Au*@Fe₃O₄) prepared in Example. Specifically, the first and second spectrometers were placed in front of and behind a UV chamber provided with a UV light source (UVP, UK) emitting light having a wavelength of 185 nm. The first differential spectrometer was used as an electrostatic particle classifying device. A fixed voltage was applied to the first differential spectrometer via a DC power supply (205B, Bertan, US) so as to extract particles having the same charge mobility in the first differential spectrometer. All of the particles (particles with the same charge mobility) leaving the first differential spectrometer were passed through a serial system consisting of an aerosol charge neutralizer (4810, HCT, Korea) and a cylindrical electrostatic precipitator to form neutral monodisperse particles (20 nm). Then, the monodisperse particles were moved to the UV chamber. Finally, the particles moved to the UV chamber via the second differential spectrometer were scanned to calculate the charge distribution corresponding to a mobility diameter which was initially selected.

FIG. 14 is a graph showing the measurement of the charge distribution of the nano complex material prepared in Example as obtained using a tandem differential spectrometer. In FIG. 14, the different peaks are caused by a difference in the charge numbers. Referring to Equation 1 described above, the ratio of the ion mobility measured by two types of differential mobility spectrometers (NDMA, 3085, TSI, USA) constituting the tandem differential spectrometer indicates the charge number (q) of the nano complex material. From FIG. 14, it was confirmed that the surface of the nano complex material of Example was positively charged.

Although not shown, after the nano complex material prepared in Example was injected into phosphate-buffered saline (PBS), the zeta potential of the nano complex material was measured using a zeta potential analyzer (Nano ZS-90, Malvern Instruments, UK). The measured zeta potential was +3.4 mV.

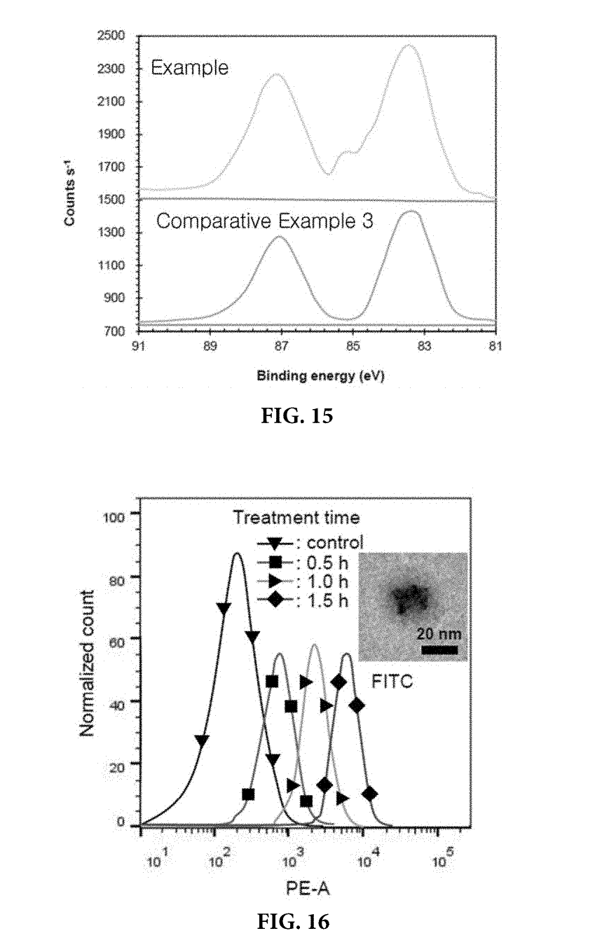

FIG. 15 is a graph showing the measurement of the Au 4f spectrum of the nano complex materials prepared in Example and Comparative Example 3 as obtained by XPS. The spectrum shows the relative binding energy value for carbon (C) 1 s (284.6 eV). The spectrum was obtained using an X-ray photoelectron spectrometer (Kratos Axis HIS). Specifically, as the light source (X-ray source) of the spectrometer, an aluminum light source that generates 1486.6 eV photons with a dwell time of 100 ms and an applied voltage of 40 eV was used. The light source was operated at 150 W while maintaining the pressure inside the spectrometer at 8 to 10 Torr. Referring to FIG. 15, unlike the nano complex material (Au@Fe₃O₄) of Comparative Example 3, the nano complex material (Au*@Fe₃O₄) of Example produced a new peak at 85.2 eV. This is due to gold (Au^{δ+}) which became positively charged.

<Cell Uptake Measurement>

HeLa cells (298 HEK) (1×10⁵ cells/well) were applied to a 12-well plate and cultured for 48 hours in order to quantitatively measure cell uptake. The nano complex material (Au*@Fe₃O₄) of Example bound with FITC at a concentration of 5 μg/mL was added to the cultured cells under conditions of 37° C. and 5% CO₂. After culturing for 60 minutes, the cells were washed with a PBS solution and collected. To measure flowing cells, the cells were dispersed in 1.0 mL of a PBS solution and measured using a FACSCalibur flow cytometer (BD Biosciences, US).

FIG. 16 is a graph of the results of the measurement of cell uptake rate of the nano complex material prepared in Example. The measurement was carried out using a fluorescence-activated cell sorter (FACS, BD Biosciences, USA). As a result, it was confirmed that the cell uptake rate of the nano complex material bound with a fluorescent labeling (FITC) was time-dependent.

<Evaluation of Photothermal Therapy>

The nano complex material (Au*@Fe₃O₄) prepared in Example was dispersed in 2% agar at concentrations of 10 μg/mL, 30 μg/mL, 50 μg/mL, 70 μg/mL, and 90 μg/mL. The agar gel was applied to a thin plastic petri dish having a 35 mm diameter to prepare samples. Using a solid-state laser system (HL7001MG, Opnext, Japan) at room temperature, samples were irradiated with a continuous laser beam (40 mW, i.e., a power density of 4.12 W/cm²) with a wavelength of 705 nm. Specifically, the sample was irradiated with a laser beam for duration times of 10 seconds, 30 seconds, and 60 seconds.

In addition, in order to evaluate the photothermal therapy performance, ATP analysis was further performed. Firefly luciferin was used as a marker to determine the level of cellular ATP.

The nano complex material (Au*@Fe₃O₄) prepared in Example was cultured for 24 hours, washed 3 times with a Hank's buffered salt solution, and then placed into a well along with 0.1 M of a CellTiter-Glo Luminescent (Promega, US) analysis reagent. Afterwards, they were mixed using an orbital shaker for 2 minutes. In addition, in order to stabilize luminescence signals, culturing was additionally carried out for 10 minutes, and luminescence was measured using Luminescence.

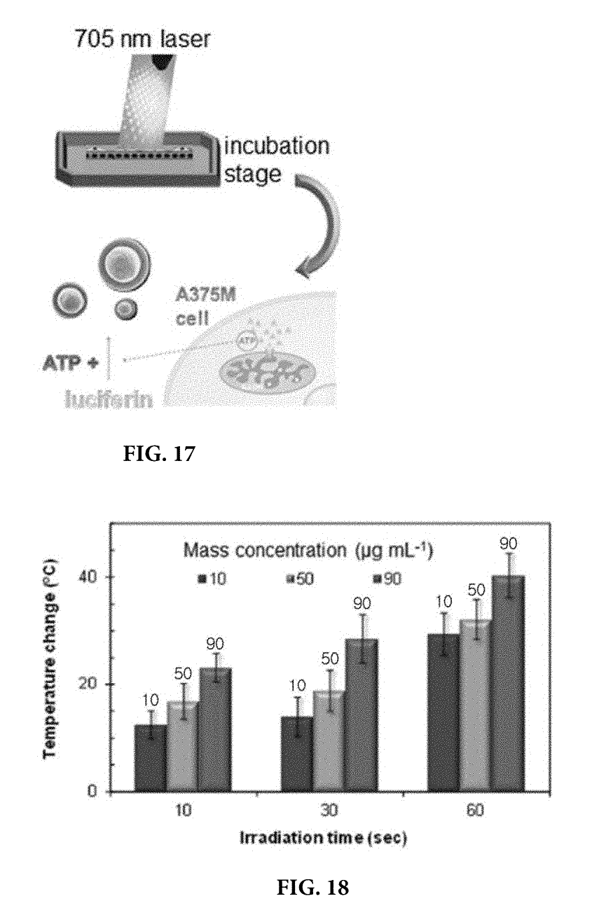

FIG. 17 is a schematic diagram of an experiment for determining the photothermal therapy performance of the nano complex material prepared in Example.

FIG. 18 is a graph of the results of measuring the photothermal therapy performance of the nano complex material prepared in Example according to the experimental method of FIG. 17.

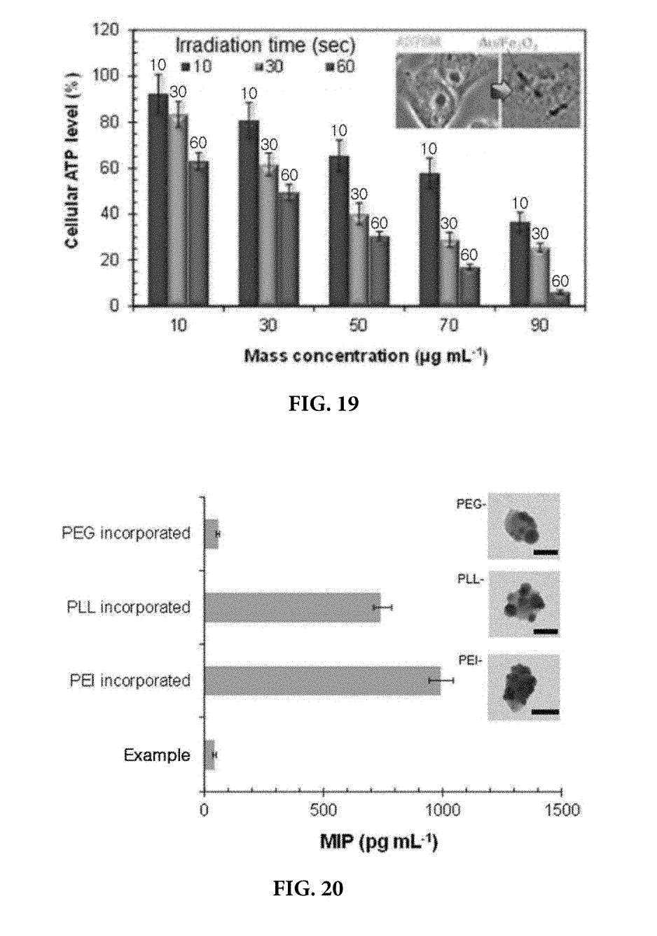

FIG. 19 is a graph showing the evaluation of the cell-killing performance of the nano complex material prepared in Example according to the experimental method of FIG. 17.

Referring to FIG. 17, in order to determine the photothermal therapy performance of the nano complex material (Au*@Fe₃O₄) prepared in Example, agar gel samples containing the nano complex material (Au*@Fe₃O₄) at varying mass concentrations were prepared. Specifically, agar gel samples containing the nano complex material (Au*@Fe₃O₄) at mass concentrations of 10 μg/mL, 50 μg/mL, and 90 μg/mL were prepared. Then, the samples were irradiated with a laser having a wavelength of 705 nm for 10 seconds, 30 seconds, and 60 seconds, and the temperature changes of the agar gel were measured using an infrared thermometer (42545, Extech, USA). As shown in FIG. 8, since the nano complex material (Au*@Fe₃O₄) of Example has a high absorption peak in the vicinity of the near-infrared ray, a laser having a wavelength of 705 nm (near-infrared ray) was selected as a light source for generating heat. Although not shown, the temperature of the agar gel without the nano complex material (Au*@Fe₃O₄) did not change. Referring to FIG. 18, the temperature change of the agar gel was the largest when the laser was emitted for 60 seconds and the nano complex material was contained at a mass concentration of 90 μg/mL, and the value of the largest temperature change was about 40.3° C. The above result suggests that the nano complex material prepared in Example can absorb the laser light and change the absorbed light into thermal energy.

Since the production of ATP is related to carbohydrate degradation of cancer cells, cell-killing performance was evaluated using an adenosine triphosphate (ATP) analysis, and the results are shown in FIG. 19. The ATP analysis was carried out using A375M cell lines (see FIG. 17), and it was determined whether laser irradiation could affect the production of ATP. Referring to the illustration in FIG. 19, it was found that the nano complex material (dark spot) prepared in Example was mostly present in the cells or on the surface. Although not shown, in the absence of the nano complex material (Au*@Fe₃O₄) prepared in Example, there was no significant change in ATP production. On the other hand, in the presence of the nano complex material (Au*@Fe₃O₄) prepared in Example, the light energy of the laser was converted to thermal energy, and the converted heat impeded ATP production of the A375M cell lines. In addition, the reduction of ATP may be due to suppression of ATP production by gene overexpression caused by the heat.

<Production of Macrophage Inflammatory Protein (MIP)>

Peritoneal macrophages were applied to a 24-well plate at a density of 1×10⁵ cells per well in 1 mL of a medium, and cultured for 24 hours. In order to adjust the particle concentration in the medium to 2 mg/mL, 0.1 mL of the sample solution containing the nano complex material of Example was injected into each well. For comparison purposes, instead of the sample solution containing the nano complex material, comparative samples containing each of polyethyleneimine (PEI, 765090, Sigma-Aldrich, US), poly-1-lysin (PLL, P4707, Sigma-Aldrich, US), and polyethylene glycol (PEG, 81188, Sigma-Aldrich) were injected into each well. After culturing for 24 hours, the supernatant of the culture medium was separated by centrifugation at 2,000 rpm for 10 minutes. Before comparing with the comparative samples, lipopolysaccharides (LPS) were added to the medium having a final concentration of 1 μg/mL. In order to measure MIP levels, the enzyme-linked immunosorbent assay (ELISA) was performed. For the above test, an MIP-2 ELISA kit (R&D Systems, USA) was used. The supernatant collected from LPS-macrophages was always diluted 10-fold before analysis. The difference was considered to be significant at p<0.01.

FIG. 20 is a graph showing the comparison of degrees of inhibition of production of macrophage inflammatory protein (MIP) of LPS-macrophage by the nano complex material prepared in Example and control groups. In the control groups, polyethylene glycol, poly-L-lysine, and polyethyleneimine were used.

Referring to FIG. 20, the nano complex material (Au*@Fe₃O₄) prepared in Example shows superior suppression of the MIP production compared to poly-L-lysine and polyethyleneimine. This trend implies that it is related to amine groups contained in poly-L-lysine and polyethyleneimine. This hypothesis is supported by the results showing excellent suppression of the MIP production even by polyethylene glycol, which does not have amine groups.

The nano complex material of the present disclosure is suitable as a biomaterial because the nano complex material has excellent light absorbance, magnetic absorbance, storage stability, drug (including genes) delivery performance, and low cytotoxicity. In particular, whereas the surface of conventional biomaterials is coated with a cationic material to improve performance, the cationic material generally has an amine group, and the amine group can bind to surface receptors of macrophages in the human body to cause inflammatory responses. On the other hand, since the surface of the nano complex material of the present disclosure is positively charged on its own, the inflammatory responses which are caused by the amine group can be suppressed.

In conclusion, the nano complex material of the present disclosure has excellent magnetic absorption, X-ray absorption, low cytotoxicity, and excellent cell-killing ability because it is prepared by combining different components and the surface thereof has been positively charged due to light irradiation. As a result, the nano complex material of the present disclosure can be appropriately used as a contrast agent for an MR-CT dual-mode imaging device and a therapeutic agent by which treatment and diagnosis are simultaneously performed.

In addition, since the nano complex material of the present disclosure does not require the conventional wet chemical formulation and the process of coating with a cationic material to be performed, inflammatory responses in the human body that may be caused by the above processes can be minimized.

1. A nano complex material, having:
a support comprising an inorganic material; and
ionic particles which are formed on the support and include a metal having a work function of 6.0 eV or less,
wherein a charge number (q) of the nano complex material as determined by Equation 1 below is in a range of 1 to 50:
$$q = \frac{{Z}_{1}}{{Z}_{2}} \qquad [ Equation 1 ]$$
wherein, in Equation 1, Z₁ represents an ionic mobility of the nano complex material, which is measured by using a first differential mobility spectrometer of a tandem differential mobility analyzer (TDMA), and Z₂ represents an ionic mobility of the nano complex material, which is measured by using a second differential mobility spectrometer.

2. The nano complex material of claim 1, wherein the inorganic material is a magnetic material, silica, or alumina.

3. The nano complex material of claim 1, wherein the metal having a work function of 6.0 eV or less is one or more selected from the group consisting of barium, silver, cadmium, aluminum, beryllium, cerium, cesium, cobalt, chromium, iron, gallium, gadolinium, hafnium, mercury, indium, magnesium, manganese, molybdenum, lead, niobium, neodymium, rubidium, rhenium, rhodium, ruthenium, scandium, tin, strontium, tantalum, terbium, tellurium, thorium, titanium, uranium, vanadium, yttrium, thallium, ytterbium, zinc, palladium, iridium, platinum, gold, and zirconium.

4. The nano complex material of claim 1, wherein the metal having a work function of 6.0 eV or less is gold (Au).

5. The nano complex material of claim 1, wherein the metal is an aggregate, and the aggregate has an average particle diameter in a range of 2 to 40 nm.

6. The nano complex material of claim 1, wherein a surface of the ionic particle is positively charged.

7. The nano complex material of claim 1, wherein the nano complex material has a peak change rate (AP) as determined by Equation 2 below in a range of 0.1 to 20 eV during X-ray photoelectron spectroscopy analysis:
ΔP=|Pₘ−Pᵢ| [Equation 2]
wherein, in Equation 2, Pₘ represents an XPS peak of the metal having a work function of 6.0 eV or less, and Pᵢ represents an XPS peak of the ionic particles including the metal having a work function of 6.0 eV or less.

8. A drug carrier, comprising:
the nano complex material according to claim 1; and
a drug supported in the nano complex material.

9. The drug carrier of claim 8, wherein the drug is one or more selected from the group consisting of an antifungal agent, an antibacterial agent, an antimicrobial agent, an antioxidant, a coolant, a soothing agent, a wound-healing agent, an anti-inflammatory agent, an anti-aging agent, an anti-wrinkle agent, a skin-lightening agent, a bleaching agent, a light-absorbing agent, a scattering agent, a skin-bleaching agent, a dye, a coloring agent, a deodorant, and a fragrance.

10. A contrast agent, comprising:
the nano complex material according to claim 1.

11. A method of preparing a nano complex material, comprising:
a forming step of forming metal particles having a work function of 6.0 eV or less;
a binding step of binding the metal particles to droplets formed by spraying a solution containing a support and a solvent; and
a light irradiating step of irradiating the droplets bound with the metal particles with ultraviolet rays having a wavelength range of 200 nm or less.

12. The method of claim 11, wherein the light irradiating step further comprises an extraction step of extracting the solvent.

13. The method of claim 11, wherein the forming step, the binding step, and the light irradiating step are carried out under a flow of an inert gas.
